(19) United States
(12) Patent Application Publication
(10) Pub. No.: US 2008/0131973 A1
(43) Pub. Date: Jun. 5, 2008

(54) Method of Analyzing Dioxins

(76) Inventors: Naotoshi Kirihara, Tokyo (JP); Norifumi Kitada, Tokyo (JP); Kenji Takahashi, Tokyo (JP); Haruaki Yoshida, Tokyo (JP); Mizuho Tanaka, Tokyo (JP); Yasuo Suzuki, Tokyo (JP)

Correspondence Address: Muramatsu & Associates, Suite 310, 114 Pacifica, Irvine, CA, 92618, US

(21) Appl. No.: 10/564,132

(22) PCT Filed: Mar. 24, 2005
(86) PCT No.: PCT/JP05/05394

§ 371 Date: Dec. 31, 2007

(30) Foreign Application Priority Data
Dec. 28, 2004 (JP) 2004-378559

(51) Int. Cl. G01N 33/00

(52) U.S. Cl. 436/139

(57) ABSTRACT

A dioxin analyzing method sensitively detecting dioxins is provided.

The method includes the first step of obtaining respective specific wavelength spectra of a plurality of dioxin isomers whose concentrations are known, selecting a plurality of specific wavelengths from each of the specific wavelength spectra, and preparing calibration curves, each showing the relationship between the ion signal intensity and the dioxin isomer concentration at any one of the selected specific wavelengths, for all the specific wavelengths selected for each dioxin isomer; the second step of preparing a sensitivity matrix showing the relationship between the ion signal intensities and the dioxin isomer concentrations at the specific wavelengths, from the calibration curves of the dioxin isomers prepared in the first step; and the third step of obtaining a specific wavelength spectrum of a sample to be analyzed, and determining the concentrations of a plurality of dioxin isomers in the sample using the ion signal intensities of the specific wavelength spectrum and the sensitivity matrix prepared in the second step.

### TECHNICAL FIELD

The present invention relates to methods for analyzing dioxins (polychlorodibenzo-para-dioxin, polychlorodibenzofuran, and coplanar polychlorobiphenyls, specified in the Law Concerning Special Measures against Dioxins) contained in gases by laser ionization mass spectrometry in real time.

### BACKGROUND ART

It has been shown that gases discharged from, for example, municipal and industrial waste incinerators, other incinerators, such as for sewage sludge, thermal cracking furnaces, and melting furnaces contain harmful organic compounds. In particular, polychlorinated dioxins and their derivatives (hereinafter collectively referred to as dioxins) are extremely toxic and a highly sensitive method of analyzing the dioxins has been desired.

The official method (JIS K 0311) for analyzing concentrations of dioxins discharged from waste incinerators or the like uses a high resolution gas chromatograph (HRGC) or a high resolution double-focusing mass spectrometer (HRMS). Although this method has been established for sensitively analyzing extremely low concentrations of dioxins, its procedure is so complicated that it takes 30 to 50 days for analysis, which is a disadvantage.

Accordingly, a prompt and highly sensitive method of dioxin analysis has been desired, and highly sensitive analytical techniques using laser light are expected to be applied to dioxin analysis.

A method has been proposed for highly sensitive analysis using laser light (for example, Non-Patent Document 1). This method measures the spectrum of chlorinated organic compounds in samples by combining supersonic jet spectrometry and laser multiphoton ionization. In this method, the spectrum is simplified by jetting the sample into a vacuum and instantaneously cooling the sample to near absolute zero.

Another method has also been proposed in which the sample is irradiated with laser light to selectively ionize a target constituent and detect the target constituent (for example, Patent Document 1).

Furthermore, a dual wavelength optical ionization mass spectrometer has been proposed which uses a second laser light with a fixed wavelength to enhance the ionization efficiency to the extent that target constituents excited into excited triplet states by a first laser light can be ionized (for example, Patent Document 2).

[Non-Patent Document 1] Rapid Commun. Mass Spectron Vol. 7, 183 (1993)

[Patent Document 1] Japanese Unexamined Patent Application Publication No. 8-222181

[Patent Document 2] Japanese Unexamined Patent Application Publication No. 2002-202289

### DISCLOSURE OF INVENTION

### Problems to be Solved by the Invention

The method disclosed in Non-Patent Document 1 has a detection limit in dioxins on the order of ppb. In order to directly analyze dioxins in exhaust gases by this method, samples need to be concentrated to 10⁵ to 10⁶ times, or the sensitivity needs to be increased to 10⁵ to 10⁶ times. Thus, it is difficult to detect such a low concentration of dioxins in practice.

In direct analysis of organic compounds containing chlorine atoms, such as dioxins, by the method disclosed in Patent Document 1, the excitation lifetime of the target constituent in an excited triplet state is reduced due to a so-called heavy atom effect as the number of chlorine atoms increases. Accordingly, the sensitivity is not sufficient, which is a disadvantage.

Patent Document 2 has explained why the method disclosed in this document enhances the ionization efficiency. Specifically, as soon as a dioxin is excited into an excited state S1 by a first laser light having a first wavelength, an internal heavy atom effect occurs and thus the excited state S1 is turned into an excited state T1 by energy transfer. Since the lifetime of the excited state T1 is on the order of microseconds and longer than the excited state S1, molecules in the excited state T1 can be efficiently ionized by irradiation of the second laser light with a second wavelength.

Thus, the method according to Patent Document 2 is on the precondition that the dioxin is irradiated with the first laser light having the first wavelength to excite the dioxin to the excited state S1.

Patent Document 2 however has not disclosed optimal wavelengths of the first laser light for changing various types of target constituents to their respective excited states from the ground states, and no other documents have taught them.

In view of the above-described disadvantages, the object of the present invention is to provide a method of sensitively analyzing dioxins without effects of coexisting substances by laser ionization mass spectrometry using supersonic jet/resonance-enhanced multiphoton ionization, even in the presence of a large amount of constituents other than target constituents.

### Means for Solving the Problems

(1) Claim 1

A method of analyzing dioxins is provided which is performed by laser ionization mass spectrometry using supersonic jet/resonance-enhanced multiphoton ionization. The method includes:

the first step of obtaining respective specific wavelength spectra of a plurality of dioxin isomers whose concentrations are known, selecting a plurality of specific wavelengths from each of the specific wavelength spectra, and preparing calibration curves, each showing the relationship between the ion signal intensity and the dioxin isomer concentration at any one of the selected specific wavelengths, for all the specific wavelengths selected for each dioxin isomer;

the second step of preparing a sensitivity matrix showing the relationship between the ion signal intensities and the dioxin isomer concentrations at the specific wavelengths, from the calibration curves of the dioxin isomers prepared in the first step; and

the third step of obtaining a specific wavelength spectrum of a sample to be analyzed, and determining the concentrations of a plurality of dioxin isomers in the sample using the ion signal intensities of the specific wavelength spectrum and the sensitivity matrix prepared in the second step.

In the first step, the specific wavelength spectra are obtained by repeating the sequence of exciting the dioxin isomers with a first laser light having a first wavelength, ionizing the excited dioxin isomers with a second laser light having a second wavelength, and measuring the intensities of ion signals, while the first wavelength of the first laser light is sequentially varied. The plurality of specific wavelengths are selected from each specific wavelength spectrum as follows:

(1) for a dioxin isomer 1,2,3,4,6,7,8-HpCDD (heptachlorodibenzo-para-dioxin), at least one specific wavelength is selected from the group consisting of 317.66 nm, 317.36 nm, 315.10 nm, 314.60 nm, 314.37 nm, 313.65 nm, 312.96 nm, 312.80 nm, 312.20 nm, 311.90 nm, 311.61 nm, 311.00 nm, 310.39 nm, and 310.12 nm;
(2) for a dioxin isomer OCDD (octachlorodibenzo-para-dioxin), at least one specific wavelength is selected from the group consisting of 321.85 nm, 321.14 nm, 319.76 nm, 317.90 nm, 316.23 nm, 315.80 nm, 315.48 nm, 315.21 nm, 314.57 nm, 312.60 nm, 312.04 nm, 311.69 nm, and 310.87 nm; and
(3) for a dioxin isomer OCDF (octachlorodibenzofuran), at least one specific wavelength is selected from the group consisting of 329.89 nm, 329.41 nm, 329.28 nm, 329.11 nm, 329.02 nm, 328.93 nm, 327.35 nm, 326.38 nm, and 325.48 nm.

In the dioxin analysis by laser ionization mass spectrometry using supersonic jet/resonance-enhanced multiphoton ionization, a dioxin in a gas jetted from a nozzle through a high-speed pulse valve into a vacuum is excited from the ground state to an excited state in an ionization zone by excitation laser light, and ionized by ionization laser light having an energy higher than or equal to the difference resulting from the subtraction of the photon energy of the excitation laser light from the ionization energy of the dioxin. The molecules of the ionized dioxin are drawn into a mass spectrometer by an electric field, and the mass spectrometer detects the signals of the ions, thus performing mass spectrometry. Examples of the mass spectrometer include time-of-flight mass spectrometers, double-focusing mass spectrometers, quadruple mass spectrometers, and ion trap mass spectrometers.

### First Step:

In the first step, a specific wavelength spectrum is obtained for each of the plurality of dioxin isomers whose concentrations are known. In this instance, the dioxin isomers are excited by a first laser light with a first wavelength. The excited dioxin isomers are ionized by a second laser light with a second wavelength and the ion signals of the isomers are measured. This procedure is repeated while the wavelength of the first laser light is sequentially varied. Specifically, the wavelength of the laser light for exciting the known concentrations of dioxin isomers are varied from 300 to 340 nm in 0.01 nm steps. Thus, specific wavelength spectra are obtained in which the horizontal axis represents the wavelength of the excitation laser light and the vertical axis represents the ion signals of the corresponding dioxin isomer excited by the excitation laser light and ionized by the ionization laser light.

FIGS. 1 to 3 show the specific wavelength spectra of three types of hepta-chlorinated and octa-chlorinated dioxin isomers obtained by use of ionization laser light with a wavelength of 213 nm and specific wavelengths in the respective specific wavelength spectra.

The intensity of the signals shown in FIGS. 1 to 3 is normalized for the corresponding isomer so that the highest intensity of the signals is 1.

After obtaining the specific wavelength spectra, a plurality of specific wavelengths are selected from each specific wavelength spectrum, preferably according to the following criteria. Wavelengths each exhibiting a high peak of ion signals are picked at wavelength intervals of 0.1 to 0.5 nm. If there are lower peaks around the picked wavelength, a wavelength exhibiting the highest peak at substantially the center of the group of the peaks is selected as a specific wavelength for dioxin forms. Thus specific wavelengths are selected from sequentially shifted wavelength regions. For furan forms, a wavelength exhibiting the highest peak in each group of the peaks to the short wavelength side is selected as a specific wavelength. The reason why the wavelength interval is set at 0.1 to 0.5 nm is that specific wavelengths can be selected even if a broad peak appears.

Calibration curves showing the relationship between the ion signal intensity and the dioxin isomer concentration are prepared for each of the specific wavelengths λ selected for each dioxin isomer, according to the following Equation 1. Although the relationship between the ion signal intensity and the dioxin concentration can be expressed by a linear equation for the sake of simplicity, it may be expressed by other functions.

S=aC+b Equation 1

### Where

S: ion signal intensity;

a: coefficient;

C: dioxin concentration; and

b: constant

Let two specific wavelengths be selected for each of the three types of dioxin isomers shown in FIGS. 1 to 3, and let the dioxin isomers in FIGS. 1 to 3 be isomers 1 to 3 respectively. Specific wavelengths λ₁ and λ₂ are selected for isomer 1, specific wavelengths λ₃ and λ₄ are selected for isomer 2, and thus specific wavelengths λ₅ and λ₆ are selected for isomer 3.

The calibration curves of dioxin isomers 1 to 3 at specific wavelengths λ₁ to λ₆ are prepared according to the following Equations 2.

$$\begin{matrix}{S}_{1} ( {λ}_{1} ) = {a}_{11} C + {b}_{11} \\ {S}_{1} ( {λ}_{2} ) = {a}_{21} C + {b}_{21} \\ ⋯ \\ ⋯ \\ {S}_{1} ( {λ}_{5} ) = {a}_{51} C + {b}_{51} \\ {S}_{1} ( {λ}_{6} ) = {a}_{61} C + {b}_{61} \\ \\ \\ {S}_{2} ( {λ}_{1} ) = {a}_{12} C + {b}_{12} \\ {S}_{2} ( {λ}_{2} ) = {a}_{22} C + {b}_{22} \\ ⋯ \\ ⋯ \\ {S}_{2} ( {λ}_{5} ) = {a}_{52} C + {b}_{52} \\ {S}_{2} ( {λ}_{6} ) = {a}_{62} C + {b}_{62} \\ \\ \\ {S}_{3} ( {λ}_{1} ) = {a}_{13} C + {b}_{13} \\ {S}_{3} ( {λ}_{2} ) = {a}_{23} C + {b}_{23} \\ ⋯ \\ ⋯ \\ {S}_{3} ( {λ}_{5} ) = {a}_{53} C + {b}_{53} \\ {S}_{3} ( {λ}_{6} ) = {a}_{63} C + {b}_{63}\end{matrix} \qquad Equations 2$$

### Second Step:

In the second step, a sensitivity matrix showing the relationship between the ion signal intensity and the dioxin isomer concentration at the plurality of specific wavelengths is prepared according to the calibration curves at the specific wavelengths of dioxin isomers prepared in the first step.

First, dioxin isomers contained in a sample to be analyzed may be identified, and the sensitivity matrix may be prepared on the basis of the identification. Alternatively, the sensitivity matrix may be prepared without the identification.

The case where dioxin isomers in a sample are identified will now be described.

The specific wavelength spectra depend on dioxin isomers, and the dioxin isomers each have a distinctive specific wavelength spectrum. Therefore, a specific wavelength spectrum of a sample containing a plurality of dioxin isomers simultaneously shows patterns of specific wavelength spectra of the dioxin isomers in the sample. The dioxin isomers in the sample can be identified by comparing the profile of the specific wavelength spectrum of the sample with previously obtained profiles of reference materials.

If two dioxin isomers 1 and 2 respectively having concentrations of C₁ and C₂ in the sample are identified, the ion signal intensities S(λ₁), S(λ₂), S(λ₃), and S(λ₄) of the sample at wavelengths λ₁, λ₂, λ₃, and λ₄ are expressed by the following simultaneous equations 3.

S(λ₁)=a₁₁C₁+a₁₂C₂+b₁

S(λ₂)=a₂₁C₁+a₂₂C₂+b₂

S(λ₃)=a₃₁C₁+a₃₂C₂+b₃

S(λ₄)=a₄₁C₁+a₄₂C₂+b₄ Equations 3

where b₁=b₁₁+b₁₂, b₂=b₂₁+b₂₂, b₃=b₃₁+b₃₂, b₄=b₄₁+b₄₂

Let the simultaneous equations be represented by a matrix, and the following equations 4 hold. The following A is referred to as the sensitivity matrix.

$$S = A C + B \\ S = [ \begin{matrix}S ( {λ}_{1} ) \\ S ( {λ}_{2} ) \\ S ( {λ}_{3} ) \\ S ( {λ}_{4} )\end{matrix} ] \\ A = [ \begin{matrix}{a}_{11} & {a}_{12} \\ {a}_{21} & {a}_{22} \\ {a}_{31} & {a}_{32} \\ {a}_{41} & {a}_{42}\end{matrix} ] \\ C = [ \begin{matrix}{C}_{1} \\ \\ {C}_{2}\end{matrix} ] \\ B = [ \begin{matrix}{b}_{1} \\ {b}_{2} \\ {b}_{3} \\ {b}_{4}\end{matrix} ] \qquad Equations 4$$

### Third Step:

In the third step, a specific wavelength spectrum of the sample are obtained, and the concentrations of the dioxin isomers in the sample are determined using the ion signal intensities of the specific wavelength spectrum and the sensitivity matrix prepared in the second step.

First, the specific wavelength spectrum of the sample is obtained by sweeping excitation laser light with wavelengths varied from 300 to 340 nm in 0.01 nm steps, in the same manner as in the first step.

For example, the above-cited dioxin isomers 1 and 2 are identified, and the ion signal intensities S(λ₁), S(λ₂), S(λ₃), and S(λ₄) of dioxin isomers 1 and 2 are measured at specific wavelengths λ₁, λ₂, λ₃, and λ₄. The concentrations C of dioxin isomers 1 and 2 in the sample are determined from the measured ion signal intensities and the sensitivity matrix.

Specifically, the concentrations C are derived from the equation C=A⁻¹ (S—B), where A⁻¹ is the inverse matrix of the sensitivity matrix A.

It has been known that the specific wavelength spectra of dioxins exhibit specific wavelengths in a wide range. If analysis is performed at all the wavelengths, enormous volumes of data are produced. By use of the calibration curves prepared at some of the specific wavelengths, the volume of data can be reduced, and a plurality of dioxins can be simultaneously and rapidly determined irrespective of the concentration.

The specific wavelengths used for the dioxin isomers can have an error of ±0.045 nm. This is because if ultra high-speed jet of gas containing dioxin isomers from a high-speed pulse valve cannot be cooled sufficiently, the peaks of ion signals at the specific wavelengths may become broad.

The error range of ±0.045 nm applies to Claims 2 to 6 as well.

### (2) Claim 2

The second step may include the sub-step of identifying dioxin isomers contained in the sample. The sensitivity matrix is prepared according to the calibration curves of the dioxin isomers identified in the sub-step.

By identifying the dioxin isomers in the sample in the second step, as mentioned in the above (1), the sensitivity matrix becomes simple, and the calculation becomes easy accordingly.

How the dioxin isomers are identified is not particularly limited, but, for example, the profiles of the known specific wavelength spectra of the dioxin isomers can be used, as described above.

### (3) Claim 3

Alternatively, in the second step, the sensitivity matrix may be prepared according to all the calibration curves prepared in the first step.

Hence, dioxin isomers in the sample are not identified before the preparation of the sensitivity matrix. This makes the sensitivity matrix complicated, but the sub-step of identifying dioxin isomers can be omitted advantageously.

For example, let the three types of dioxin isomers 1 to 3 in the sample have concentrations C₁ to C₃, respectively. A matrix equation, equation 5, holds using the ion signal intensities S(λ₁) to S(λ₆) at wavelengths λ₁, to λ₆. The other process steps for determination can be performed in the same manner as in the above procedure including the identification.

$$S = A C + B \\ S = [ \begin{matrix}S ( {λ}_{1} ) \\ S ( {λ}_{2} ) \\ … \\ … \\ S ( {λ}_{5} ) \\ S ( {λ}_{6} )\end{matrix} ] \\ A = [ \begin{matrix}{a}_{11} & {a}_{12} & {a}_{13} \\ {a}_{21} & {a}_{22} & {a}_{23} \\ … & … & … \\ … & … & … \\ {a}_{51} & {a}_{52} & {a}_{53} \\ {a}_{61} & {a}_{62} & {a}_{63}\end{matrix} ] \\ C = [ \begin{matrix}{C}_{1} \\ {C}_{2} \\ {C}_{3}\end{matrix} ] \\ B = [ \begin{matrix}{b}_{1} \\ {b}_{2} \\ … \\ … \\ {b}_{5} \\ {b}_{6}\end{matrix} ] \qquad Equation 5$$

### (4) Claim 4

The present invention is also directed to another method of analyzing dioxins which identifies a dioxin isomer from a specific wavelength spectrum obtained by laser ionization mass spectrometry using supersonic jet/resonance-enhanced multiphoton ionization.

The method includes: the first step of obtaining a specific wavelength spectrum of a sample to be analyzed; and the second step of identifying 1,2,3,4,6,7,8-HpCDD (heptachlorodibenzo-para-dioxin) contained in the sample according to the specific wavelength spectrum obtained in the first step and specific wavelengths of 1,2,3,4,6,7,8-HpCDD (heptachlorodibenzo-para-dioxin) obtained in advance. The specific wavelength spectrum is obtained in the first step by repeating the sequence of exciting the sample with a first laser light having a first wavelength, ionizing the excited sample with a second laser light having a second wavelength, and measuring the intensity of ion signals, while the first wavelength of the first laser light is varied step by step. In the second step, at least two specific wavelengths are selected from the specific wavelengths of 1,2,3,4,6,7,8-HpCDD (heptachlorodibenzo-para-dioxin) shown in the following table, and it is determined whether the selected specific wavelengths of 1,2,3,4,6,7,8-HpCDD are shown in the specific wavelength spectrum of the sample obtained in the first step.

TABLE 4
1,2,3,4,6,7,8-HpCDD (heptachlorodibenzo-para-dioxin)

| | Specific wavelength (nm) |
|---|---|
| 1 | 317.66 |
| 2 | 317.36 |
| 3 | 315.10 |
| 4 | 314.60 |
| 5 | 314.37 |
| 6 | 313.65 |
| 7 | 312.96 |
| 8 | 312.80 |
| 9 | 312.20 |
| 10 | 311.90 |
| 11 | 311.61 |
| 12 | 311.00 |
| 13 | 310.39 |
| 14 | 310.12 |

### (5) Claim 5

The present invention is also directed to another method of analyzing dioxins which identifies a dioxin isomer from a specific wavelength spectrum obtained by laser ionization mass spectrometry using supersonic jet/resonance-enhanced multiphoton ionization.

The method includes: the first step of obtaining a specific wavelength spectrum of a sample to be analyzed; and the second step of identifying OCDD (octachlorodibenzo-para-dioxin) contained in the sample according to the specific wavelength spectrum obtained in the first step and specific wavelengths of OCDD (octachlorodibenzo-para-dioxin) obtained in advance. The specific wavelength spectrum is obtained in the first step by repeating the sequence of exciting the sample with a first laser light having a first wavelength, ionizing the excited sample with a second laser light having a second wavelength, and measuring the intensity of ion signals, while the first wavelength of the first laser light is varied step by step. In the second step, at least two specific wavelengths are selected from the specific wavelengths of OCDD (octachlorodibenzo-para-dioxin) shown in the following table, and it is determined whether the selected specific wavelengths of OCDD are shown in the specific wavelength spectrum of the sample obtained in the first step.

TABLE 5
OCDD (octachlorodibenzo-para-dioxin)

| | Specific wavelength (nm) |
|---|---|
| 1 | 321.85 |
| 2 | 321.14 |
| 3 | 319.76 |
| 4 | 317.90 |
| 5 | 316.23 |
| 6 | 315.80 |
| 7 | 315.48 |
| 8 | 315.21 |
| 9 | 314.57 |
| 10 | 312.60 |
| 11 | 312.04 |
| 12 | 311.69 |
| 13 | 310.87 |

### (6) Claim 6

The present invention is also directed to another method of analyzing dioxins which identifies a dioxin isomer from a specific wavelength spectrum obtained by laser ionization mass spectrometry using supersonic jet/resonance-enhanced multiphoton ionization.

The method includes: the first step of obtaining a specific wavelength spectrum of a sample to be analyzed; and the second step of identifying OCDF (octachlorodibenzofuran) contained in the sample according to the specific wavelength spectrum obtained in the first step and specific wavelengths of OCDF (octachlorodibenzofuran) obtained in advance. The specific wavelength spectrum is obtained in the first step by repeating the sequence of exciting the sample with a first laser light having a first wavelength, ionizing the excited sample with a second laser light having a second wavelength, and measuring the intensity of ion signals, while the first wavelength of the first laser light is varied step by step. In the second step, at least two specific wavelengths are selected from the specific wavelengths of OCDF (octachlorodibenzofuran) shown in the following table, and it is determined whether the selected specific wavelengths of OCDF are shown in the specific wavelength spectrum of the sample obtained in the first step.

TABLE 6
OCDF (octachlorodibenzofuran)

| | Specific wavelength (nm) |
|---|---|
| 1 | 329.89 |
| 2 | 329.41 |
| 3 | 329.28 |
| 4 | 329.11 |
| 5 | 329.02 |
| 6 | 328.93 |
| 7 | 327.35 |
| 8 | 326.38 |
| 9 | 325.48 |

### Advantages

According to one of the aspects of the present invention, hepta-chlorinated an octa-chlorinated dioxin isomers contained in a sample can be precisely and simultaneously determined by laser ionization mass spectrometry using supersonic jet/resonance-enhanced multiphoton ionization.

Also, according to the other aspects of the present invention, it can be correctly examined whether a sample containing a plurality of dioxin isomers contains a specific dioxin isomer.

### BRIEF DESCRIPTION OF THE DRAWINGS

FIG. 1 shows a specific wavelength spectrum of 1,2,3,4,6,7,8-HpCDD (heptachlorodibenzo-para-dioxin) and selectable specific wavelengths in the spectrum.

FIG. 2 shows a specific wavelength spectrum of OCDD (octachlorodibenzo-para-dioxin) and selectable specific wavelengths in the spectrum.

FIG. 3 shows a specific wavelength spectrum of OCDF (octachlorodibenzofuran) and selectable specific wavelengths in the spectrum.

FIG. 4 is a schematic diagram of the structure of a laser ionization mass spectrometry system used in a method of analyzing dioxins according to an embodiment of the present invention.

FIG. 5 is a specific wavelength spectrum of a sample used in an embodiment of the present invention.

### BEST MODE FOR CARRYING OUT THE INVENTION

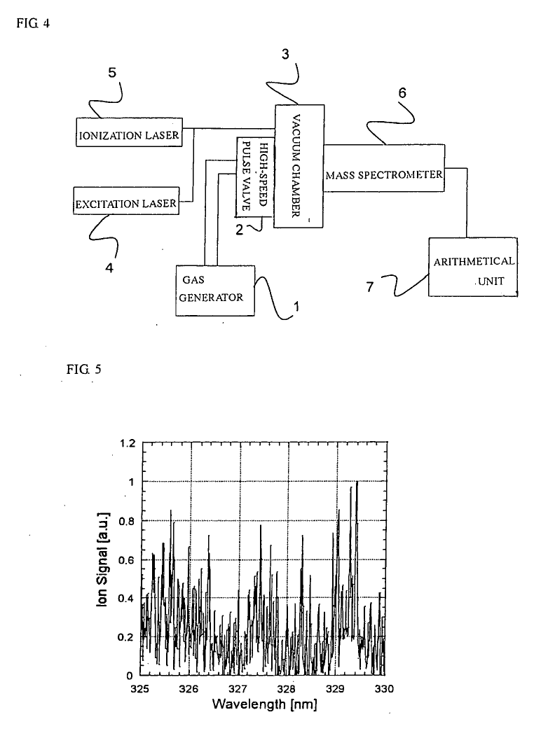

FIG. 4 is a schematic diagram of the structure of a laser ionization mass spectrometry system used in a method of analyzing dioxins according to an embodiment of the present invention. The laser ionization mass spectrometry system will be described below with reference to FIG. 4.

Dioxins contained in carrier gas generated from a gas generator 1 are delivered with the carrier gas to a high-speed pulse valve 2 and jetted into a vacuum chamber 3 from a nozzle to be cooled. The dioxins in the carrier gas jetted from the nozzle are excited and ionized in an ionization zone with excitation laser light emitted from a tunable laser oscillator 4 and ionization laser light emitted from an ionization laser oscillator 5.

The ionized dioxins are drawn into a mass spectrometer 6 (reflectron time-of-flight mass spectrometer) by an attractive electrostatic field generated between a repeller electrode and an extraction electrode. Specifically, the dioxin ions accelerated by an attractive electric field are further accelerated and pulse-compressed by an attractive electric field generated between an extraction electrode and a grounding electrode. The ions that have passed by the grounding electrode are focused in the diameter direction perpendicular to their traveling direction by an electrostatic field of an einzel lens. Then, the ions are deviated from the orbit by an electric field at a deflecting electrode. The ions that have passed by the deflecting electrode are introduced into the mass spectrometer 6 through a differential exhaust opening. The ionized dioxins in the mass spectrometer 6 are deviated from the orbit to arrive at an ion detector by an ion reflecting electrode, and are converted to electrical signals. The signals are data-processed in an arithmetical unit 7.

The gas generator 1, which may be, for example, a high-boiling-point organic standard gas generator manufactured by Gastec Corporation, supplies constant concentrations of dioxins to the high-speed pulse valve.

The high-speed pulse valve 2 preferably has a nozzle of, for example, 1.1 mm in diameter. The nozzle temperature is preferably 200° C. or more from the viewpoint of preventing the adsorption of the dioxins.

The vacuum chamber 3 contains a multiple reflection device that increases the sensitivity by multiply reflecting laser light and accumulating the laser light in the ionization zone. The multiple reflection device includes two mirror sets opposing each other in the horizontal direction. The mirror sets each include a plurality of concave mirrors arranged in a ring.

The tunable laser oscillator 4 for exciting dioxins emits nanosecond pulsed laser light, and may be a dye laser oscillator or an optical parametric oscillator. In order to excite dioxins selectively, it is preferable that the spectral line width of the excitation laser light be 0.01 nm or less.

The excitation laser light has an energy of about 1 mJ. In order to prevent fragmentation resulting from excessive laser intensity, the excitation laser light irradiating the dioxins is not focused with a lens or the like.

The ionization laser oscillator 5 is a Nd:YAG laser oscillator, and nanosecond pulsed laser light quintupled (to a wavelength of 213 nm) is used as the ionization laser light. In order to prevent one-color two-photon ionization by the quintuple light, it is preferable that the ionization laser light have an energy of 0.1 mJ or less. The ionization laser light, as well as the excitation laser light, is not focused by a lens or the like.

The excitation laser light and the ionization laser light are synchronized by a delay pulse generator and superficially turned into an apparent single laser beam in a laser light mixer. The two types of laser light are emitted into a vacuum and, in the ionization zone, simultaneously irradiate the dioxins in a carrier gas jetted into the vacuum.

An embodiment of the method of analyzing dioxins using the above-described system will now be described. In the present embodiment, gas containing 1,2,3,4,6,7,8-HpCDD (heptachlorodibenzo-para-dioxin), OCDD (octachlorodibenzo-para-dioxin), and OCDF (octachlorodibenzofuran) is used as a sample to be analyzed.

A plurality of dioxin isomers including known concentrations of 1,2,3,4,6,7,8-HpCDD (heptachlorodibenzo-para-dioxin), OCDD (octachlorodibenzo-para-dioxin), and OCDF (octachlorodibenzofuran) are swept with excitation laser light at wavelengths varied from 300 to 340 nm in 0.01 nm steps. Thus, the specific wavelength spectra of the dioxin isomers are obtained.

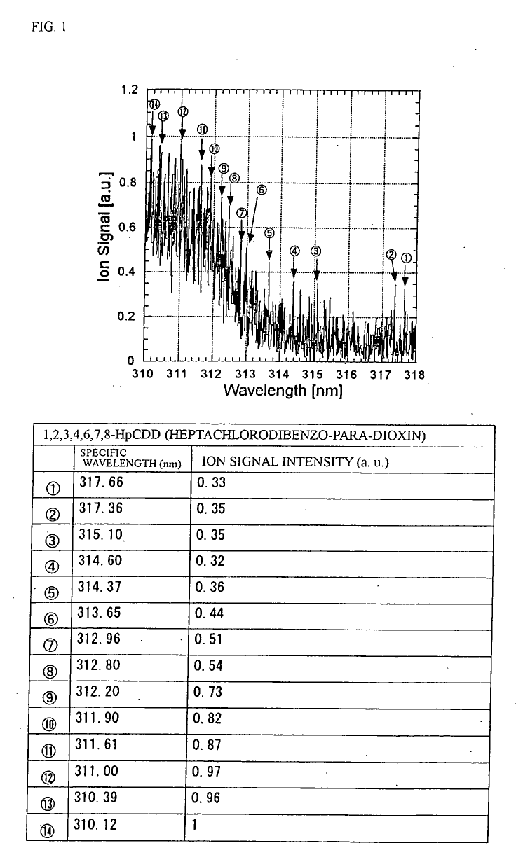

FIGS. 1 to 3 show the obtained specific wavelength spectra of 1,2,3,4,6,7,8-HpCDD (heptachlorodibenzo-para-dioxin), OCDD (octachlorodibenzo-para-dioxin), and OCDF (octachlorodibenzofuran), respectively.

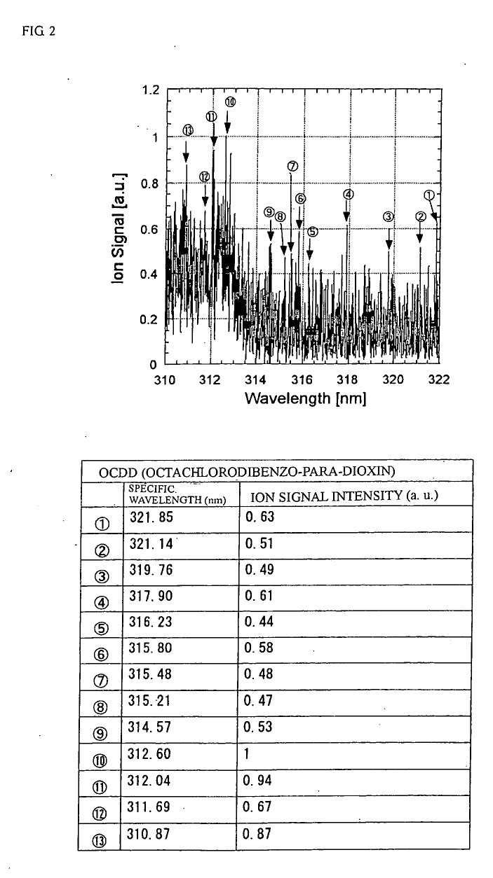

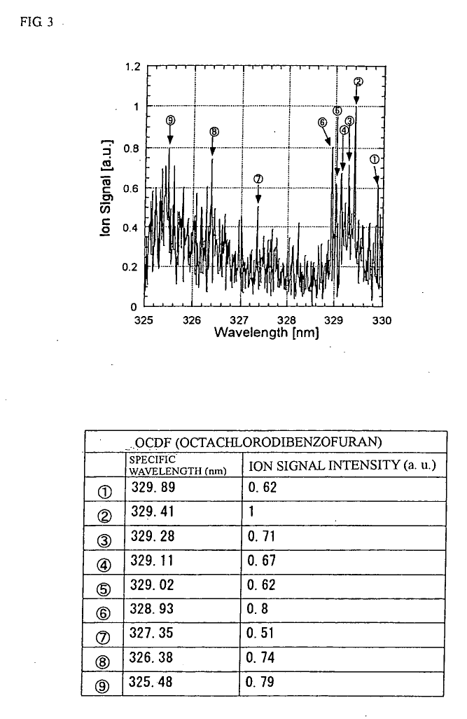

Specific wavelengths of λ₁=317.66 nm and λ₂=317.36 nm are selected for 1,2,3,4,6,7,8-HpCDD (heptachlorodibenzo-para-dioxin) from the specific wavelength spectrum shown in FIG. 1; specific wavelengths of λ₃=321.85 nm and λ₄=321.14 nm are selected for OCDD (octachlorodibenzo-para-dioxin) from the specific wavelength spectrum shown in FIG. 2; and specific wavelengths of λ₅=329.89 nm and λ₆=329.41 nm are selected for OCDF (octachlorodibenzofuran) from the specific wavelength spectrum shown in FIG. 3.

Then, calibration curves each showing the relationship between the ion signal intensity and the dioxin isomer concentration at any one of the selected specific wavelengths λ₁, λ₂, λ₃, λ₄, λ₅, and λ₆ are prepared for all the selected specific wavelengths selected for each dioxin isomer.

When the concentration of 1,2,3,4,6,7,8-HpCDD (heptachlorodibenzo-para-dioxin) is C₁ (ppt), the concentration of OCDD (octachlorodibenzo-para-dioxin) is C₂ (ppt), and the concentration of OCDF (octachlorodibenzofuran) is C₃ (ppt), the calibration curves at the respective wavelengths are expressed as follows:

S_{1234678DD}(λ₁)=4C₁+0.02;S_{123467BDD}(λ₂)=5C₁+0.02

S_{OCDD}(λ₃)=5C₂+0.05;S_{OCDD}(λ₄)=2C₂+0.01

S_{OCDF}(λ₅)=2C₃+0.04;S_{OCDF}(λ₆)=4C₃+0.04

These calibration curves have been obtained in advance and stored in a database.

On the premise above, a sample containing 1,2,3,4,6,7,8-HpCDD (heptachlorodibenzo-para-dioxin), OCDD (octachlorodibenzo-para-dioxin), and OCDF (octachlorodibenzofuran) is swept with the excitation laser light at wavelengths varied from 300 to 340 nm in 0.01 nm steps. Thus, the specific wavelength spectrum of the sample is obtained. The obtained specific wavelength spectrum of the sample is shown in FIG. 5.

If it has not been known what dioxin isomers are contained in the sample, they are identified from the specific wavelength spectrum of the sample and previously obtained specific wavelength spectra of reference materials. Specifically, the specific wavelength spectrum of the sample is checked for the specific wavelengths of the reference materials one by one from its longer wavelength side, and dioxin isomers having the same specific wavelengths as the sample are identified. For example, the specific wavelength spectrum of a dioxin isomer exhibiting a specific wavelength of λ₁=329.89 nm as in the specific wavelength spectrum of the sample is referred to. As shown in FIG. 3, OCDF (octachlorodibenzofuran) has the specific wavelength of 329.89 nm. The specific wavelength spectrum of OCDF shows another specific wavelength of 329.41 nm in addition to 329.89 nm. Then, the specific wavelength spectrum of the sample is checked for specific wavelengths, and it is found that the sample has the specific wavelength of 329.41 nm. Thus, OCDF (octachlorodibenzofuran) in the sample is identified.

In the same manner, 1,2,3,4,6,7,8-HpCDD (heptachlorodibenzo-para-dioxin) and OCDD (octachlorodibenzo-para-dioxin) are identified.

After the identification of 1,2,3,4,6,7,8-HpCDD (heptachlorodibenzo-para-dioxin), OCDD (octachlorodibenzo-para-dioxin), and OCDF (octachlorodibenzofuran) in the sample, a sensitivity matrix is prepared from the previously prepared calibration curves for determining the concentrations of the 1,2,3,4,6,7,8-HpCDD (heptachlorodibenzo-para-dioxin), the OCDD (octachlorodibenzo-para-dioxin), and the OCDF (octachlorodibenzofuran).

In the present embodiment, dioxin isomers contained in the sample have already been identified. Accordingly, a calibration curve at a wavelength is selected from each of the above calibration curves of 1,2,3,4,6,7,8-HpCDD (heptachlorodibenzo-para-dioxin), OCDD (octachlorodibenzo-para-dioxin), and OCDF (octachlorodibenzofuran), and these calibration curves are used for preparing the sensitivity matrix. For example, when calibration curves at λ₁, λ₃, and λ₅ are used, the following simultaneous equations hold for the sensitivity matrix:

S_{123467BDD}(λ₁)=4C₁+0.02

S_{OCDD}(λ₃)=5C₂+0.05

S_{OCDF}(λ₅)=2C₃+0.04

The simultaneous equations are expressed by the following equations 6 in matrixes.

$$S = A C + B \\ S = [ \begin{matrix}S ( {λ}_{1} ) \\ S ( {λ}_{2} ) \\ S ( {λ}_{3} )\end{matrix} ] A = [ \begin{matrix}4 & 0 & 0 \\ 0 & 5 & 0 \\ 0 & 0 & 2\end{matrix} ] \\ C = [ \begin{matrix}{C}_{1} \\ {C}_{2} \\ {C}_{3}\end{matrix} ] B = [ \begin{matrix}0.02 \\ 0.05 \\ 0.04\end{matrix} ] \qquad Equations 6$$

Hence, the inverse matrix A⁻¹ of the sensitivity matrix A is expressed by equation 7.

$${A}^{- 1} = [ \begin{matrix}0.25 & 0 & 0 \\ 0 & 0.2 & 0 \\ 0 & 0 & 0.5\end{matrix} ] \qquad Equation 7$$

S(λ₁), S(λ₃), and S(λ₅) are determined by measuring the ion signal intensities of the sample at λ₁=317.66 nm, λ₃=321.85 nm, λ₅=329.89 nm. Let S(λ₁)=50 a.u., S(λ₃)=100 a.u., and S(λ₅)=120 a.u., and then C₁=12.5 (ppt), C₂=20 (ppt), and C₃=60 (ppt).

It is thus shown that the sample contains 12.5 ppt of 1,2,3,4,6,7,8-HpCDD (heptachlorodibenzo-para-dioxin), 20 ppt of OCDD (octachlorodibenzo-para-dioxin), and 60 ppt of OCDF (octachlorodibenzofuran).

1. A method of analyzing dioxins by laser ionization mass spectrometry using supersonic jet/resonance-enhanced multiphoton ionization, the method comprising:
the first step of obtaining respective specific wavelength spectra of a plurality of dioxin isomers whose concentrations are known, selecting a plurality of specific wavelengths from each of the specific wavelength spectra, and preparing calibration curves, each showing the relationship between the ion signal intensity and the dioxin isomer concentration at any one of the selected specific wavelengths, for all the specific wavelengths selected for each dioxin isomer;
the second step of preparing a sensitivity matrix showing the relationship between the ion signal intensities and the dioxin isomer concentrations at the specific wavelengths, from the calibration curves of the dioxin isomers prepared in the first step; and
the third step of obtaining a specific wavelength spectrum of a sample to be analyzed, and determining the concentrations of a plurality of dioxin isomers in the sample using the ion signal intensities of the specific wavelength spectrum of the sample and the sensitivity matrix prepared in the second step,
wherein in the first step, the specific wavelength spectra are obtained by repeating the sequence of exciting the dioxin isomers with a first laser light having a first wavelength, ionizing the excited dioxin isomers with a second laser light having a second wavelength, and measuring the intensities of ion signals, while the first wavelength of the first laser light is varied step by step, and the plurality of specific wavelengths are selected from each specific wavelength spectrum according to the following (1) to (3):
(1) for a dioxin isomer 1,2,3,4,6,7,8-HpCDD (heptachlorodibenzo-para-dioxin), at least one specific wavelength is selected from the group consisting of 317.66 nm, 317.36 nm, 315.10 nm, 314.60 nm, 314.37 nm, 313.65 nm, 312.96 nm, 312.80 nm, 312.20 nm, 311.90 nm, 311.61 nm, 311.00 nm, 310.39 nm, and 310.12 nm;
(2) for a dioxin isomer OCDD (octachlorodibenzo-para-dioxin), at least one specific wavelength is selected from the group consisting of 321.85 nm, 321.14 nm, 319.76 nm, 317.90 nm, 316.23 nm, 315.80 nm, 315.48 nm, 315.21 nm, 314.57 nm, 312.60 nm, 312.04 nm, 311.69 nm, and 310.87 nm; and
(3) for a dioxin isomer OCDF (octachlorodibenzofuran), at least one specific wavelength is selected from the group consisting of 329.89 nm, 329.41 nm, 329.28 nm, 329.11 nm, 329.02 nm, 328.93 nm, 327.35 nm, 326.38 nm, and 325.48 nm.

2. The method according to claim 1, wherein the second step includes the sub-step of identifying dioxin isomers contained in the sample, and the sensitivity matrix is prepared according to the calibration curves of the dioxin isomers identified in the sub-step.

3. The method according to claim 1, wherein the sensitivity matrix is prepared according to all the calibration curves prepared in the first step.

4. A method of analyzing dioxins which identifies a dioxin isomer from a specific wavelength spectrum obtained by laser ionization mass spectrometry using supersonic jet/resonance-enhanced multiphoton ionization, the method comprising:
the first step of obtaining a specific wavelength spectrum of a sample to be analyzed by repeating the sequence of exciting the sample with a first laser light having a first wavelength, ionizing the excited sample with a second laser light having a second wavelength, and measuring the intensity of ion signals, while the first wavelength of the first laser light is varied step by step; and
the second step of identifying 1,2,3,4,6,7,8-HpCDD (heptachlorodibenzo-para-dioxin) contained in the sample from the specific wavelength spectrum of the sample obtained in the first step and specific wavelengths of 1,2,3,4,6,7,8-HpCDD (heptachlorodibenzo-para-dioxin) obtained in advance, by selecting at least two specific wavelengths from the specific wavelengths of 1,2,3,4,6,7,8-HpCDD (heptachlorodibenzo-para-dioxin) shown in Table 1 and determining whether the selected specific wavelengths are shown in the specific wavelength spectrum of the sample obtained in the first step.
TABLE 1
1,2,3,4,6,7,8-HpCDD (heptachlorodibenzo-para-dioxin)

| | Specific wavelength (nm) |
|---|---|
| 1 | 317.66 |
| 2 | 317.36 |
| 3 | 315.10 |
| 4 | 314.60 |
| 5 | 314.37 |
| 6 | 313.65 |
| 7 | 312.96 |
| 8 | 312.80 |
| 9 | 312.20 |
| 10 | 311.90 |
| 11 | 311.61 |
| 12 | 311.00 |
| 13 | 310.39 |
| 14 | 310.12 |

5. A method of analyzing dioxins which identifies a dioxin isomer from a specific wavelength spectrum obtained by laser ionization mass spectrometry using supersonic jet/resonance-enhanced multiphoton ionization, the method comprising:
the first step of obtaining a specific wavelength spectrum of a sample to be analyzed by repeating the sequence of exciting the sample with a first laser light having a first wavelength, ionizing the excited sample with a second laser light having a second wavelength, and measuring the intensity of ion signals, while the first wavelength of the first laser light is varied step by step; and
the second step of identifying OCDD (octachlorodibenzo-para-dioxin) contained in the sample from the specific wavelength spectrum of the sample obtained in the first step and specific wavelengths of OCDD (octachlorodibenzo-para-dioxin) obtained in advance, by selecting at least two specific wavelengths from the specific wavelengths of OCDD (octachlorodibenzo-para-dioxin) shown in Table 1 and determining whether the selected specific wavelengths are shown in the specific wavelength spectrum of the sample obtained in the first step.
TABLE 2
OCDD (octachlorodibenzo-para-dioxin)

| | Specific wavelength (nm) |
|---|---|
| 1 | 321.85 |
| 2 | 321.14 |
| 3 | 319.76 |
| 4 | 317.90 |
| 5 | 316.23 |
| 6 | 315.80 |
| 7 | 315.48 |
| 8 | 315.21 |
| 9 | 314.57 |
| 10 | 312.60 |
| 11 | 312.04 |
| 12 | 311.69 |
| 13 | 310.87 |

6. A method of analyzing dioxins which identifies a dioxin isomer from a specific wavelength spectrum obtained by laser ionization mass spectrometry using supersonic jet/resonance-enhanced multiphoton ionization, the method comprising:
the first step of obtaining a specific wavelength spectrum of a sample to be analyzed by repeating the sequence of exciting the sample with a first laser light having a first wavelength, ionizing the excited sample with a second laser light having a second wavelength, and measuring the intensity of ion signals, while the first wavelength of the first laser light is varied step by step; and
the second step of identifying OCDF (octachlorodibenzofuran) contained in the sample from the specific wavelength spectrum of the sample obtained in the first step and specific wavelengths of OCDF (octachlorodibenzofuran) obtained in advance, by selecting at least two specific wavelengths from the specific wavelengths of OCDF (octachlorodibenzofuran) shown in Table 1 and determining whether the selected specific wavelengths are shown in the specific wavelength spectrum of the sample obtained in the first step.
TABLE 3
OCDF (octachlorodibenzofuran)

| | Specific wavelength (nm) |
|---|---|
| 1 | 329.89 |
| 2 | 329.41 |
| 3 | 329.28 |
| 4 | 329.11 |
| 5 | 329.02 |
| 6 | 328.93 |
| 7 | 327.35 |
| 8 | 326.38 |
| 9 | 325.48 |
